(19) United States
(12) Patent Application Publication
(10) Pub. No.: US 2012/0271289 A1
(43) Pub. Date: Oct. 25, 2012

(54) HAIR REMOVAL DEVICES AND METHODS

(75) Inventors: Shimon Eckhouse, Haifa (IL); Tuvia-Dror Kutscher, Shoham (IL); Amit Meridor, Moshav Bet Shearim (IL); Danny Ayre, Nevel Monson (IL)

(73) Assignee: Syneron Medical Ltd., Yokneam Lllit (IL)

(21) Appl. No.: 12/665,777

(22) PCT Filed: Jan. 22, 2008
(86) PCT No.: PCT/US08/00859

§ 371 Date: May 14, 2012

Related U.S. Application Data
(60) Provisional application No. 60/881,486, filed on Jan. 22, 2007.
(60) Provisional application No. 60/936,739, filed on Jun. 22, 2007.

(51) Int. Cl. A61B 18/20; A61B 18/02; A61B 17/50; A61B 18/18

(52) U.S. Cl. 606/9; 606/41; 606/33; 606/20; 606/133

(57) ABSTRACT

We describe devices, methods, and systems used for hair removal. In particular, we describe hair removal devices, particularly epilation devices, methods of using those devices, and systems including those hair removal devices. Our hair removal devices include, in combination, a.) at least one primary energy source that applies that energy, e.g., radio-frequency (RF), high intensity focused ultrasonic (HIFU) energy, or high intensity light, e.g., intense-pulsed light (IPL) or light from flash lamps or lasers, to the skin or to hair in a continuous, semi-continuous, or pulsed mode and b.) a hair removal component or components, such as rotary mechanical hair removal structures or epilators, that perform a mechanical hair removal step. Auxiliary treatment or control components such as comparatively lower power heaters, ultrasound devices, coolers, impedance measurement devices, etc. may also be included in the combination or used in conjunction with our combination device.

### RELATED APPLICATIONS

This application derives benefit from provisional application No. 60/881,486, filed Jan. 22, 2007 and provisional application No. 60/936,739, filed Jun. 22, 2007, the entirety of which are incorporated by reference for all purposes.

### FIELD

We describe devices, methods, and systems used for hair removal. In particular, we describe hair removal devices, particularly epilation devices, methods of using those devices, and systems including those hair removal devices. Our hair removal devices include, in combination, a.) at least one primary energy source that applies that energy, e.g., radio-frequency (RF), high intensity focused ultrasonic (HIFU) energy, or high intensity light, e.g., intense-pulsed light (IPL) or light from flash lamps or lasers, to the skin or to hair in a continuous, semi-continuous, or pulsed mode and b.) a hair removal component or components, such as rotary mechanical hair removal structures or epilators, that perform a mechanical hair removal step. Auxiliary treatment or control components such as comparatively lower power heaters, ultrasound devices, coolers, impedance measurement devices, etc. may also be included in the combination or used in conjunction with our combination device.

### BACKGROUND

Hair removal devices are commonly divided into two general groups: depilators and epilators. Depilators are devices for removal of hair at or above the skin surface, usually by cutting the hair or by weakening the hair and then removing it. Depilation creams, waxes, and lotions, e.g., those marketed under the VEET and VANIQA marks (together taken by some definitions as a subgroup of depilators) also remove hair by weakening the hair and then removing it. Epilators are devices that pull or pluck hair, including the portion of the hair below the skin surface.

Depilators may be, for instance, electric or manual shavers. Shavers are based on the use of a sharp blade cutting the hair. So-called electric shavers cut hair at high speed with the blade approximately perpendicular to the hair. Manual shavers utilize blades operating at a low angle to the skin. Shaving methods have the advantage of leaving very smooth skin; however, the main disadvantage of shaving is that hair grows back right away.

The epilator group of devices include devices such as electric tweezers and hair pulling mechanisms. Plucking devices provide a longer lasting hair removal effect than the depilators, but hair nonetheless usually grows back. Plucking devices may pull all of a hair structure or, in some instances because a hair is brittle or the squeeze of the tweezer is too tight, pull only a part of the hair, e.g., the hair above the skin surface. Other disadvantages are that the hair must be sufficiently long to be grasped and this method can be quite painful. Advantages of these mechanical devices are that they are useful on all skin types and colors and on all hair types and colors.

Hair may be permanently removed by destroying the papilla at the base of the hair within the hair follicle. One method for destroying the papilla is known as electrolysis. It is usually applied manually, hair by hair. In the electrolysis procedure, a direct current resulting from a direct voltage (often at 200 to 500 volts) is applied to the papilla at the base of each hair through an appropriate probe needle. The current is applied for a relatively long period of time. Application of direct current causes the disassociation of water molecules making up a large percentage of the total composition of the papilla. Electrolysis, however, is in disfavor since it is a painful process requiring an experienced user and that the treatment be on a hair-by-hair basis.

Another epilation method, known as electro-coagulation, that destroys the papillum, hair-by-hair, uses a high frequency RF probe needle. The RF epilator probe needle often employs a blunt or bulbous point and is inserted into the follicle a short distance to warm that follicle tissue. The tip is blunt to avoid penetrating the follicle wall and to avoid puncturing a capillary. The effectiveness of the process and amount of energy necessary to remove the hair depends in large measure upon the size of the hair and the moisture content of the skin in the vicinity of the hair. Since individual hair size and localized skin moisture content will vary, the procedure may be ineffective ultimately due to impedance mismatch between the electro-coagulator and the hair and local skin. Further, this procedure is also considered to be painful and suffers from the same type of disadvantages as does electrolysis.

U.S. Pat. No. 4,224,944, to Wallace, modifies the shape of the electro-coagulation device to lessen the pain of the procedure and to improve the effectiveness of the step. U.S. Pat. No. 4,372,315, to Shapiro et al, measures the impedance of the treatment site during an RF pulse to adjust the length of the pulse and thereby attempt to lessen pain and improve effectiveness.

Still another manual epilation procedure, shown in U.S. Pat. No. 2,888,927, to Fozard, replaced the electro-coagulation needle with a pair of tweezers. In this procedure, a pair of tweezers is used to grasp each hair. The tweezers apply a high voltage, apparently DC, to the hair shaft. Hair is a poor conductor of electricity and the results were spotty.

U.S. Pat. Nos. 4,566,454, and 5,364,394, to Mehl et al, show the improvement of adjusting the RF frequency to match the impedance of the hair. Other Mehl patents, U.S. Pat. Nos. 4,174,713, 5,470,332, and 5,864,252 show other improvements to the manual tweezer RF device and method.

Other improvements to this procedure involving the addition of an ionic fluid to the skin during the period that the tweezers are used to pull the hair, is shown in U.S. Pat. No. 4,498,474, to Chalmers et al and treatment of the hair with a conductive material, is shown in U.S. Pat. No. 5,364,394, to Mehl.

A laser-based analog to the manual tweezer methods and devices is found in U.S. Pat. No. 3,538,919, to Mayer, U.S. Pat. No. 4,388,924, to Weissman et al, and U.S. Pat. No. 4,617,926, to Sutton. These procedures are used to treat one hair at a time.

Alleviation of pain during these manual epilation procedures is a recurring theme. U.S. Pat. No. 4,646,735, to Seney, shows the use of refrigeration, localized to the region of the treatment area, to cool the skin and vastly improve the procedure's comfort. The patent also provides background on the concept of cooling surgical instruments.

U.S. Pat. No. 4,813,412, to Yamazaki et al, shows a manual epilation device that measures a variety of physical skin and body parameters to optimize the RF or DC treatment pulse for a particular hair.

U.S. Pat. No. 6,544,259, to Tsaliovich, discloses a manual, tweezer-based epilation device that utilizes an ultrasound source and an RF source to treat the hair prior to pulling it from the skin.

Another distinct class of epilators, often sold as personal appliances, is illustrated by U.S. Pat. No. 5,190,559, to Gabion et al. This patent shows an epilation device made up of, in essence, a collection of flexible pinching members for grasping and extracting hairs from a user. Adjacent pairs of these springy pinching members are oscillated side-to-side, or quickly and repetitively pushed together and then pulled apart using motor-driven actuating bars. This pinching and release cycle is quite short in length and, during the pinching portion of the cycle, a pair of these adjacent pinching members capture and squeeze a hair to be plucked or pulled from the user. As the device is moved along the skin, that hair is pulled and finally extracted from the skin along with its below-skin components. The device employs a number of these paired pinching members, e.g., up to twelve pairs or so, allowing it to remove multiple individual hairs simultaneously.

Another variation of this class of personal-use epilators is shown in U.S. Pat. No. 4,575,902, to Alazet, U.S. Pat. No. 4,960,422, to Demeester, and U.S. Pat. No. 5,207,689, to Demeester. Unlike the flexible, but non-rotating pinching members used in the Gabion et al device discussed above, these epilators employ a number of flexible, quickly rotating, disc-shaped, pinch members. During rotation, a pair of adjacent discs is pushed together to grasp an individual hair and, as rotation continues, to pull the hair from the skin, desirably with its below-skin components. The hairs to be removed extend through an opening in the epilator case to the periphery of the rotating blades for their extraction. This class of devices also employs a number of paired rotating discs allowing simultaneous and continuous removal of multiple individual hairs.

U.S. Pat. No. 5,849,018, to Rosson et al, shows a personal-use epilator appliance with a moistening and cooling component, e.g., a sprayer or roller laying down fluids such as water or alcoholic solutions, and an evaporator to evaporate the applied fluid and to lower the skin temperature. This cooling component is said to partially numb the skin and to help alleviate pain normally associated with epilation procedures.

U.S. Pat. No. 6,261,301, to Knesh et al, shows a personal-use epilator appliance having a pain reduction feature made up of high voltage sparking electrodes that cause a comparatively low level pain to the epilation site just prior to the actual plucking of the hair. The concept is that the pain from the spark is lower and blanks the potential later pain.

Another class of hair removal devices and methods involving the application of light energy to the hair and are based on the thermal effects occurring as a result of the application of pulsed light energy, in the infrared or near infrared spectrum, causing permanent damage to the elements of the hair root and follicle responsible for hair growth or re-growth. The objective is to selectively damage hair without damaging the skin. Such light may be applied to large areas of the skin and consequently remove many hairs at once. However, these energy-based hair removal methods, at least the ones used to treat large areas of skin in every pulse, may require multiple treatments (in the range of 5 to 10) for hair removal and have limited permanency. Also, these light energy-based have the significant disadvantage, compared to mechanical methods, of being more or less effective depending upon hair and skin color and/or type.

In some prior light-based epilation procedures, the hair shaft functions as the pathway for transmission of light energy transmission towards the hair root. This transmitted light energy is intended to heat and to injure the lower parts of the hair responsible for hair growth. One drawback to this procedure is this: the hair shaft, due to its melanin content, may be darker than the surrounding skin and therefore absorb most of the light and, indeed, may be preferentially warmed or even burnt. If the hair shaft is burnt, it does not function as a suitable light pathway to the hair root. Thus, although the hair shaft is burnt and is removable, the relative permanence of the treatment is compromised. Further, at the conclusion of a light-based hair removal procedure, a hair shaft may remain in the skin and comes out several days later. This result is not aesthetically pleasing.

Adjunct RF sources have been added to certain prior light-based hair removal devices to lessen the devices' reliance on hair-skin contrast. One such device is commercially known as elos™. The efficacy of RF is independent of skin-hair contrast. The addition of an RF energy source to a light-based device lowers the burn risks of the device in that it lowers the fluence of light required for an efficient procedure. However, it does not totally eliminate the dependence of the combination process on the hair shaft-skin contrast since the passageway of the applied light to the root of the hair is the hair shaft. The hair shaft must be in place for the combination procedure to be effective. Moreover, the RF is generally applied to the skin surface. That RF energy should be incident on the hair root, well below the skin surface, for effective long-term hair removal. Application of the RF energy to the skin surface tends to concentrate the concentration of that energy near the skin surface rather than in deeper tissues.

None of the cited patents and published patent applications disclose the devices described and claimed herein.

### SUMMARY

Described are devices, methods, and systems for hair removal. The devices, methods, and systems include at least one primary energy source, e.g., RF, HIFU, and light sources, utilized in combination with mechanical hair removal components, devices, means, or methods for mechanical hair removal. The primary energy sources may be used to apply energy to the skin or hair in a pulsed, semi- or quasi-continuous, or continuous manner. Of special interest as the mechanical hair removal component of our combination device, are rotary-style epilators having multiple pairs of high speed rotating wheels, perhaps enclosed in a shielding drum, where each pair pinches or (tweezes) an individual hair and extracts that hair during that rotation. The multiple rotating wheels simultaneously remove multiple hairs during operation.

We also describe auxiliary or secondary components that may be included in combination in our devices. Such auxiliary or secondary components include comparatively lower energy heaters, light sources, and ultrasound emitters; coolers, skin impedance measurement devices, and the like.

In certain of our described devices and procedures—specifically those utilizing light sources as the primary energy source in combination with mechanical epilators—we may illuminate only a small fraction of the skin, specifically an area surrounding the rotating tweezers, thereby providing specific targeting of hair shaft and follicle. Since, when the hair shaft is pulled, the lower regions of the follicle are ephemerally closer to the light source, the light is not as scattered at the lower follicle structure as it would be if the hair were not pulled. This focus may cause a reduction in pain level since only small part of skin is illuminated. Further, the immediate cosmetic effect is enhanced over earlier procedures since the portion of the hair shaft below the skin surface (the “invisible” hair) is removed during the procedure. In most conventional light-based professional hair removal systems, the invisible portion of the hair shaft falls out several days later. However, as noted elsewhere here, the sources may apply the energy over a broader area with still excellent results.

Similarly, our combination devices employing HIFU as the primary energy source, may be operated in such a way that the ultrasonic source or sources are energized as the hair shaft is pulled upwardly toward the skin surface. Those sources are focused at the lower end of the hair shaft or applied through the hair shaft.

Our combination epilator, including a mechanical epilator component and at least one primary energy source component, pulls the hair root towards the upper layers of the skin. In this dermal region, the concentration of applied RF or light energy is higher, the focus of the HIFU is specific.

Although secondary in relative importance to our disclosed combination primary energy source-mechanical epilator, certain other combinations of our disclosed components in combination with other skin treatment devices (e.g., depilators including, razors, etc.) or materials (e.g., depilation creams and waxes) are also useful. That is to say: a combination of at least one primary energy source such as a light source, HIFU device, RF source or combinations of these components, is also suitable for conducting hair removal procedures. Additionally, we have observed that applying the disclosed primary energy sources subsequently (and immediately to a skin area) to a mechanical epilation step may be beneficial since any blood ephemerally remaining in the follicle opening or in the tissue adjacent the follicle opening as a result of the epilation, provides sites for absorbing light and RF energy. Skin redness, also likely caused by the physical removal of the hair, is evidence of further adjacently situated blood. As noted elsewhere, blood is a superior receptor of RF compared to the neighboring tissue, thus theoretically concentrating the RF around the follicle and in effect targeting the follicle. Treating the follicular sites containing blood often also inhibits or delays later hair growth associated with those sites.

Finally, several ancillary or auxiliary components may be added to our combination epilator. Cooling or chilling components may be included in our combinations to provide a cooling function and to alleviate or to reduce initial pain from the mechanical epilation step. Ultrasound emitters, skin warmers (e.g., lower power light, heat, or RF sources) may be included to soften the skin or otherwise ease the epilation step. Components to measure physical parameters of the treatment site (e.g., skin impedance or temperature) may also be added.

### BRIEF DESCRIPTION OF THE DRAWINGS

FIG. 1 is a cross-sectional drawing of the outer layers of the skin showing the details of a single hair site.

FIG. 2 is a depiction of the growth cycle of a hair.

FIGS. 3A-3D show various views of an example of a rotary mechanical epilator suitable as the mechanical component of our epilator.

FIG. 4 shows a perspective view of another example of a rotary mechanical epilator suitable as the mechanical component of our epilator.

FIGS. 5A to 7B show schematic views for the placement of RF electrodes adjacent mechanical rotary epilator blades in our combination RF-mechanical epilator.

FIGS. 8A-8C show various schematic circuits for the RF component of our combination RF-mechanical epilator.

FIG. 9 shows the conceptual operation of our RF-mechanical epilator.

FIGS. 10A-10B show schematic views for the placement of light sources adjacent mechanical rotary epilator blades our combination light-mechanical epilator.

FIG. 11 shows the conceptual operation of our light-mechanical epilator.

FIGS. 12A-12B show schematic views for the placement of high intensity focused ultrasound sources with respect to mechanical rotary epilator blades in our combination HIFU-mechanical epilator.

FIG. 13 shows a schematic view for the placement of light sources and RF sources adjacent mechanical rotary epilator blades in our combination RF-light-mechanical epilator.

FIGS. 14 and 15 show photos of examples of the use of our epilators.

### DESCRIPTION

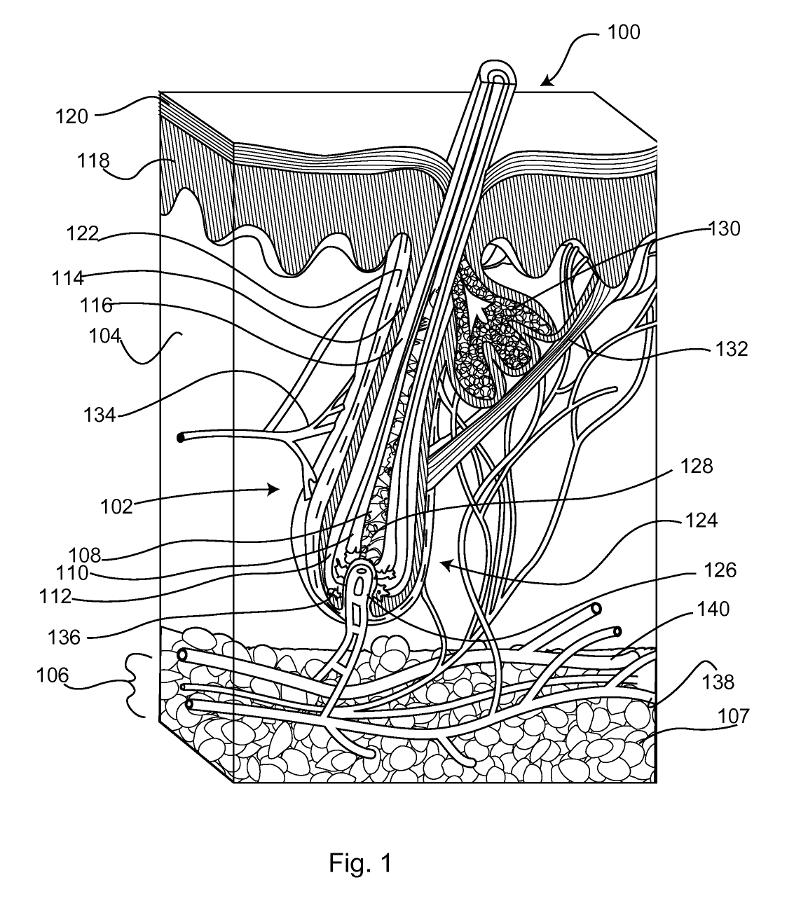

FIG. 1 shows a cross-section of the outer layers of the skin and of a hair. To understand the utility of our devices and procedures, some understanding of the anatomy of the hair is desirable.

A hair is made up of columns of dead, self-adhering, keratinized cells. The shaft (100) is the visible portion of the hair extending beyond the skin surface. The root (102) of the hair is the portion of the hair below the skin surface that penetrates into the dermis (104) and often into the subcutaneous layer (106) with its component adipose tissue (107). The shaft (100) and the root (102) of the hair are made up of three components; a.) the innermost medulla (108)—made up of two or three layers of cells containing pigment granules and air spaces, b) the cortex (110) forming the major portion of the hair made up of elongated cells that contain pigment granules and air spaces, and c.) the outermost layer, the cuticle (112) of the hair, made up of a single layer of thin, flat, heavily keratinized cells resembling shingles.

Surrounding the root (102) of the hair is the hair follicle made up of the external root sheath (114) and the internal root sheath (116). The external root sheath (114) is a downward continuation of the dermis (104), which, in turn is made up of the stratum basale (118) and the stratum corneum (120); near the surface, the external root sheath (114) contains all of the epidermal layers. At the base of the hair follicle, the external root sheath (114) is only made up of the stratum basale (118). The internal root sheath (116) forms a cellular sheath between the external root sheath (114) and the hair. There is further connective root tissue (122) between the external root sheath (114) and the dermis (104).

At the base of each follicle is the bulb (124). This structure houses an indentation, the papilla (126) of the hair, which contains areolar connective tissue. The bulb (124) also contains a region of cells, the matrix (128) which is the germinal layer of the hair. The papilla (126) also contains many blood vessels branching from the arterial vasculature (138) and to the venous vasculature (140). The cells of the matrix (128) derive from the stratum basale (118) and are responsible for the growth of existing hairs and produce new hairs when older hairs are shed. Matrix (128) cells are also responsible for the cells of the internal root sheath (116).

Also shown in FIG. 1 are sebaceous glands (130) and smooth, arrector pili muscle bundles (132). The arrector pili muscle (132) extends from the superficial dermis of the skin to the side of the external root sheath (114). Under various stimuli, autonomic nerves (142) stimulate the arrector pili muscle (132) to contract thereby pulling the hair shaft (100) into a vertical position and to form so-called “goosebumps” around the hair.

Around each follicle are nerve endings, hair root plexuses (134), that are sensitive to touch, i.e., when a hair shaft (100) is moved.

The color of hair is due primarily to melanin, synthesized by melanocytes (136) located in the matrix (128) of the bulb (124) and passed into the cells of the cortex (110) and medulla (108).

Sebaceous glands (130) or oil glands are typically connected to hair follicles. The secreting portions of the glands lie in the dermis (104) and open into the necks of hair follicles or directly onto a skin surface. The glands secrete an oily substance called sebum that is a mixture of cholesterol, proteins, fats, inorganic salts, and pheromones. Sebum coats the surface of hairs and prevents them from drying and becoming brittle. Additionally, sebum prevents excessive evaporation of water from the skin and maintains its suppleness.

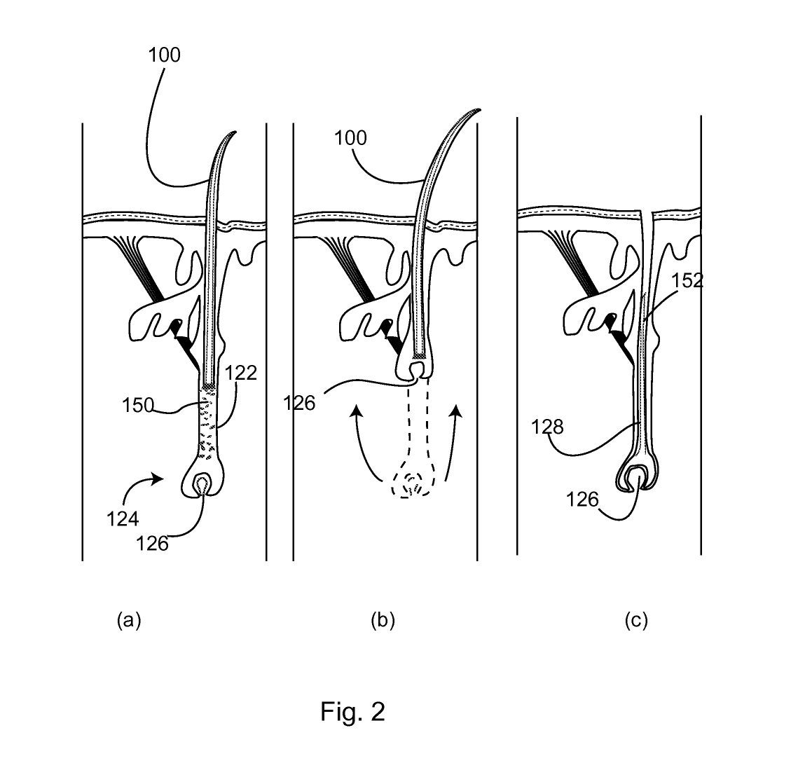

FIG. 2 shows the cycle of growth of an individual hair. Panel (a) of FIG. 2 shows the “catagen” stage, in which the hair shaft (100) loses its mooring and begins to exit the hair follicle, i.e., the external root sheath (114) and internal root sheath (116) as seen more clearly in Fig.l. The connective root tissue (122) pulls the bulb (124) with the included dermal papilla (126) upward. The dying cells (150) in the matrix (128 in FIG. 1) are depicted in panel (a).

Panel (b) of FIG. 2 shows the “telogen” phase during which upward movement of the derma papilla (126) and the additional movement of the old hair (100) shaft occurs. The shaft (100) may fall out during this phase or may later fall out.

Panel (c) of FIG. 2 shows the “anagen” phase during which the derma papilla (126) causes the matrix (128) to rebuild the follicle, i.e., the external root sheath (114) and the internal root sheath (116) seen in FIG. 1, as well as a new hair (152).

Combination High Energy-Mechanical Epilator

Our device comprises a combination of a mechanical epilator, typically a rotary epilator as described below, and one or more primary, high energy sources suitable for harming or injuring the hair follicular region during, or closely adjacent to, the mechanical epilating step. The primary, high energy sources may comprise radio-frequency (RF) sources, high intensity focused ultrasonic (HIFU) energy sources, or high intensity light sources, e.g., intense-pulsed light (IPL) or light from flash lamps or lasers. The primary energy source may be continuously energized or pulsed, perhaps, but not necessarily, in coordination with the pulling of the hair and lifting the skin surface. The energy may be focused at the follicular region of the hair as it is pulled from the skin. The energy may applied to be more diffuse; in that when applied in the region of the hair, the energy passes through the nearby tissue to the follicular region. Similarly, the energy may be applied temporally just before or just after the extension of the hair by the mechanical epilator component, either by timing the application of the energy to the skin such that the energy application is not simultaneous with hair extension or by placement of the energy focus adjacent the mechanical epilation site.

Our combination device may further comprise auxiliary or secondary components such as comparatively lower energy thermal heaters, RF sources, light sources, and ultrasound emitters; coolers; and temperature or skin impedance measurement devices.

Combination RF-Mechanical Epilator

In general, we have found that our combination RF/mechanical epilator devices and the procedures for using those devices are most effective for removing hair (in the sense that after use of the RF device, the so-treated region remains substantially free of visible hair for longer periods of time) when the hair follicle and the hair bulb are pulled or moved toward the surface of the skin causing relatively more extensive treatment with the applied RF energy. That is to say: the more highly irradiated are the follicle and the hair bulb with applied RF, the more pronounced are the cosmetic effects, e.g., reduction in hair density or growth rate and absence of hair for lengthy periods.

Similar qualitative results may be had with our combination light-based/mechanical epilator devices, combination HIFU/mechanical epilator devices, and procedures as discussed below. The primary light-based energy-applying components and HIFU-energy applying components may be used in combination with the RF-applying component or in isolation.

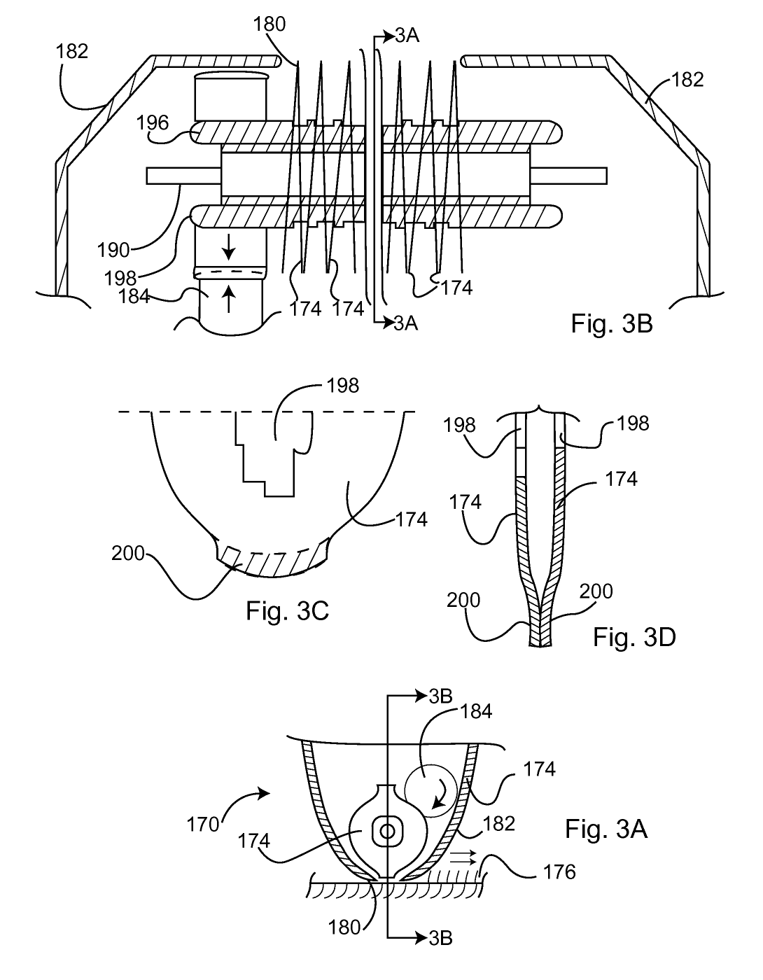

FIGS. 3A-3D show one variation of a mechanical epilator having rotary blades that pinch hair and extract the hair as the blade rotates. This style of epilator may form the mechanical portion of our combination epilator.

FIG. 3A shows a schematic partial side view of the mechanical epilator (170) with rotating pinching blades (174). The pinching action of the rotating blades is better shown in FIG. 3B. The blades interact with hair (176) on skin (178) by pinching that hair (176) through a slot or opening (180) in guard or housing (182). A drive gear (184) for turning the rotary pinch blades (174) is also shown.

FIG. 3B shows a partial cutaway, front view of the FIG. 3A epilator. The multiple rotary blades (174) are shown to pinch together in the vicinity of the opening (180) in housing (182). Drive gear (184) rotates shaft (190) and, consequently, blades (174). A pair of locator bars (196, 198) press alternate blades (174) towards each other causing those alternating blades (174) to pinch at opening (180). The blades (174) may either be deflected or rotated towards a neighboring blade to accomplishing the pinching action. This pinching continues as the blades continue to rotate, pulling the pinched hair from the skin. Upon further rotation, the pinching relaxes thereby releasing the then-extracted hair. Because of the multiple blades, the rotary epilator removes or operates upon multiple blades simultaneously.

FIG. 3C shows a partial side-view of one blade (174) with a drive region (198) and a radially extended pinching region (200). FIG. 3D shows a cross-section of a pair of blades (174) as seen in FIG. 3C, with drive region (198) and pinch regions (200). The pinch regions (200) in FIG. 3D are depicted to be in the general position that would be found as the blades (174) pinch the hair and, as the blades continue to rotate, extract and then release the hair.

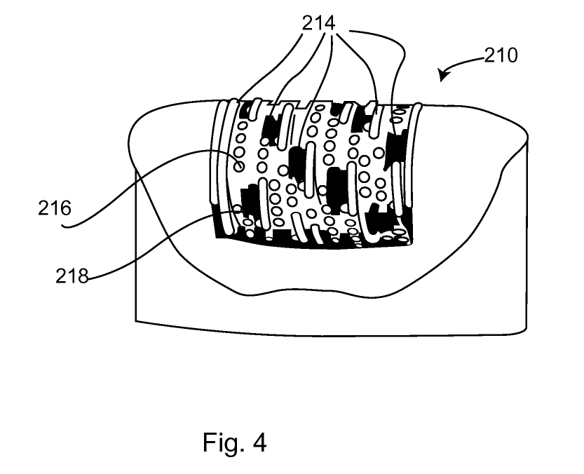

FIG. 4 is a perspective view of the removable head of a BRAUN epilator. The head (210) has been removed from a drive section that would contain, e.g., a drive motor, drive gears, batteries, on-off switch.

In this device, the pinch blades (214) rotate coincidently with (and within) a drum (216) and the blade pinch regions extend through openings (218) in that drum (216). Small stubs on the drum (216) surface are expected to position hair shafts for enhanced hair extraction.

Typical of such epilators are the BRAUN SILK-EPIL epilator and the devices shown in U.S. Pat. Nos. 5,190,559; 6,287,190; and 6,669,704, the entirety of which are incorporated by reference.

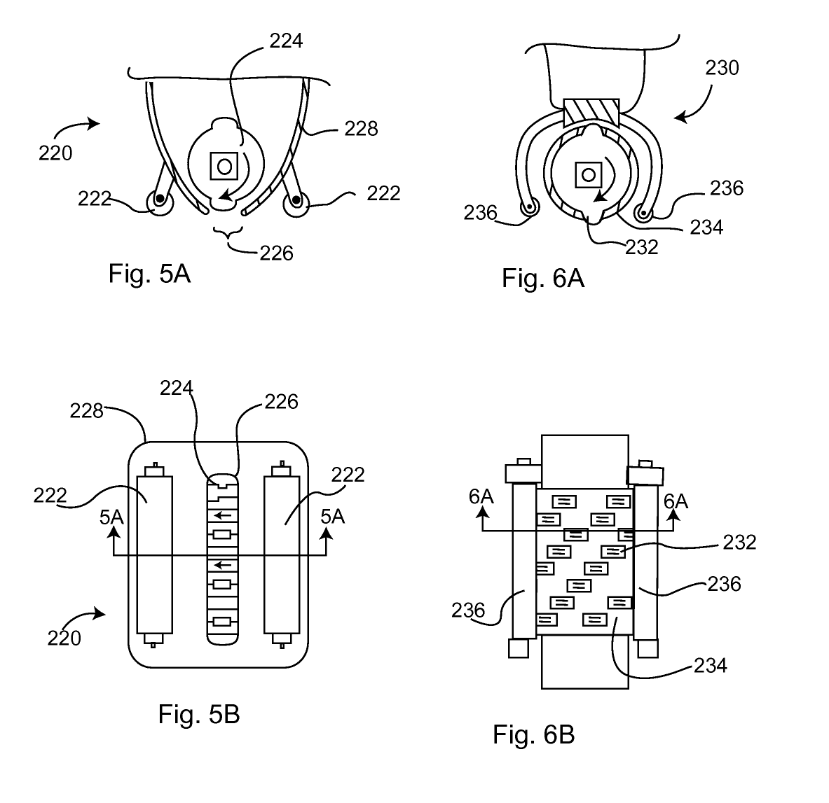

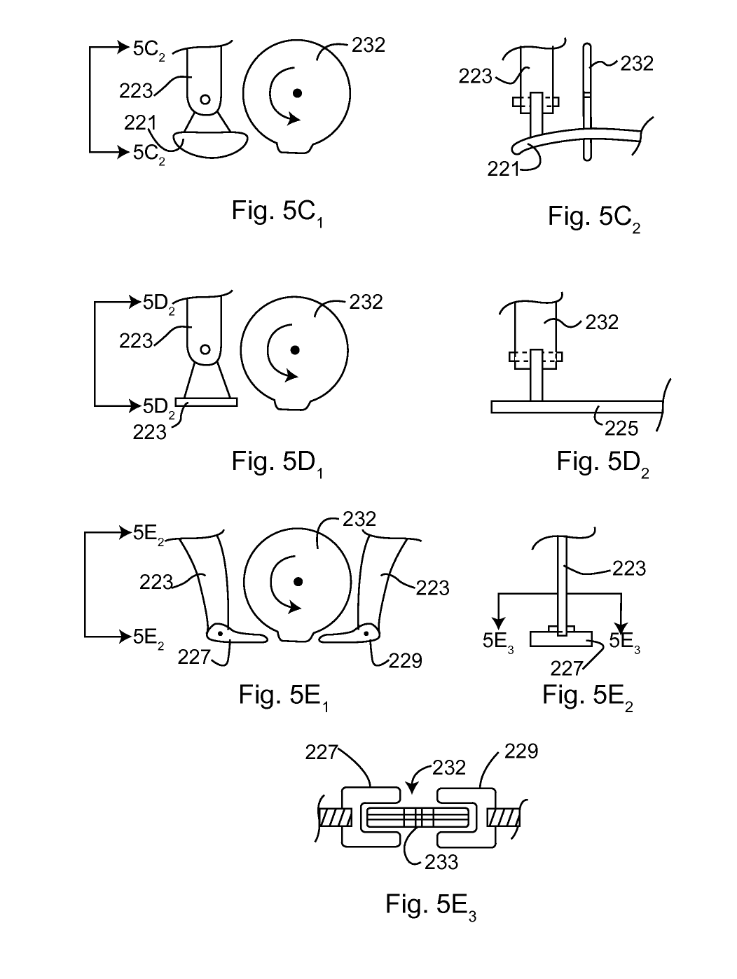

FIGS. 5A to 7B show examples of an appropriate RF electrode placement adjacent rotary pinch drums. The RF electrodes are shown to be rollers to allow or to facilitate movement of the combination epilator over the skin during an epilation treatment. The RF electrodes may, of course, be of a different configuration. For instance, the electrodes may be fixed (or non-rolling) with respect to the epilator blades and have a flat or curved contact surface with the skin. In any case, the electrodes will typically have a conductive skin contact surface, that may be metallic, but other contact surfaces appropriate for delivering RF to the skin are suitable. For instance, the contact surfaces may be coated with a polymeric coating where the nature of the applied RF (e.g., the RF frequency or the applied power level) does not require direct conduction through the skin for application of that power to the partially “pulled” hair follicle and bulb.

FIG. 5A shows a variation of our combination RF mechanical epilator (220), in partial side-view cross section, having a pair of roller electrodes (222) in leading-trailing positions to the mechanical pinching epilator blades (224) extending through opening (226) in case (228). The variation shown in FIGS. 5A and 5B includes a mechanical epilator assembly of the type discussed above with regard to FIGS. 3A-3D.

FIG. 5B shows a bottom view of the FIG. 5A device (220) and also shows placement of the roller electrodes (222) with respect to the epilator pinch blades (224) extending through opening (226). The relative placement of the electrodes (224), e.g., their distance from the site of the pinched and pulled hair and the shape of the electrode (e.g., flat, curved, roller, varying in shape and integrated into regions more closely adjacent the sides of the individual blade pinch points, etc.), may be varied as desired to achieve specific design goals. It should be recognized that no electrode shape will be optimum for all design goals. For instance, placing electrodes closer to the point at which the hair is pulled by the rotary epilator blade will likely make the device harder for the user to manipulate, in that the device will be more limited in the breadth of the angle of engagement to the skin. Other design compromises will result in different electrode configurations.

FIG. 5C₁ shows a partial side view of a stationary electrode (221) having a rounded configuration that is attached to the epilator body by a bracket (223). The electrode (221) is rounded to allow ease of movement across the skin to be treated. A rotating epilator blade (232) is shown to allow visualization of the relative positioning of the electrode (221) to the rotating epilator blade (232). FIG. 5C₂ shows a partial front view of the electrode (221) and the rotating epilator blade (232). The electrode (221) has a gentle curve allowing conformance to the skin, for instance, a limb.

FIG. 5D₁ shows a partial side view of a stationary electrode (225) having a relatively straight configuration that is attached to the epilator body by a bracket (223). A rotating epilator blade (232) is also shown.

FIG. 5D₂ shows a partial front view of the electrode (225) and the rotating epilator blade (232). The electrode (225) is relatively straight and maintains a large surface area with the skin.

The variations shown in FIGS. 5C₁ to 5D₂ may include an electrode situated on the opposite side of the rotating epilator blade (232) in the manner shown in FIGS. 5A, 5E₁, and 5E₃.

FIG. 5E₁ shows a partial side view of a pair of stationary electrodes (227, 229) each having a partially surrounding or “foot” configuration. The electrodes (227, 229), as shown with clarity in partial top view in FIG. 5E₃, partially surround the region wherein the rotating blade (232) pinch the hair in extracting it from the skin. Electrode (227) and electrode (229) may be at the same RF potential, both delivering RF energy to the skin, and a return electrode, perhaps remote, completing the circuit. Electrode (227) and electrode (229) may form a complete circuit, one delivering RF energy to the skin and the other functioning as a return electrode completing the circuit.

FIG. 5E₂ shows a partial front view of the electrode (227) and the rotating epilator blade (232).

FIG. 6A shows a partial, cutaway side-view of another variation of our combination RF-mechanical epilator device (230) having epilator blades (232) that rotate within a drum (234). This mechanical epilator component section is of the type shown in FIG. 4. In this variation, the roller electrodes (236) are also in a leading-trailing relationship to the epilator blades (232). Again, the electrodes (236) may have other shapes and spacing.

FIG. 6B shows a bottom view of the device shown in FIG. 6A. The roller electrodes (236) may be seen in relationship to the epilator blades (232) that rotate within drum (234).

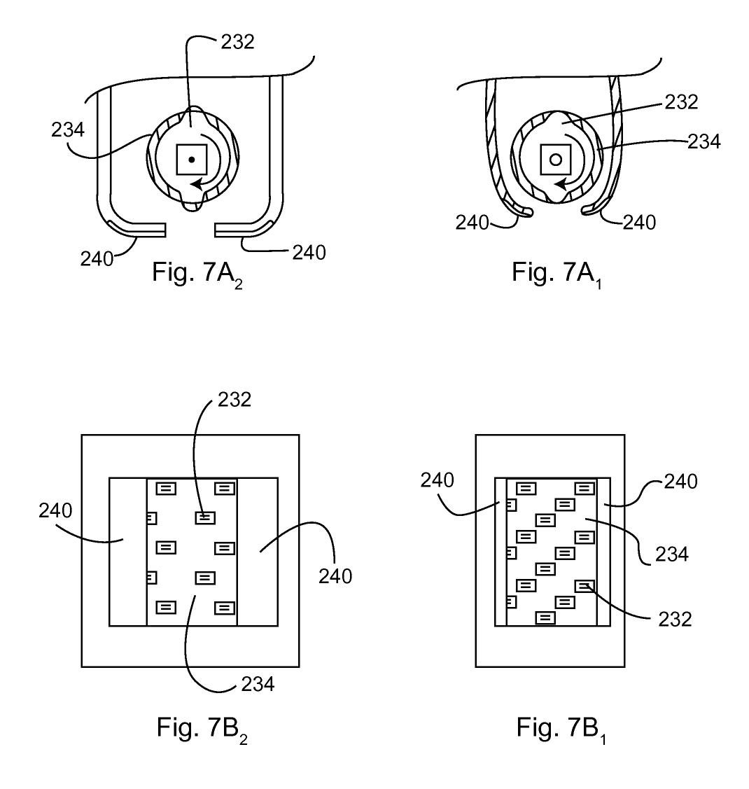

FIG. 7A shows a partial, cutaway side-view of still another variation of our combination RF-mechanical epilator device (238) having epilator blades (232) that rotate within a drum (234). This mechanical epilator component section is of the type shown in FIG. 4. In this variation, the electrodes (240) comprise curved sheets in a leading-trailing relationship to the epilator blades (232). The electrodes (240) are integrated into epilator case (242). These electrodes (240) may form the two points of RF passage through the user's body. The two electrodes may also be held at the same potential with another electrode on the body completing the electrical circuit.

FIG. 7B1 shows a bottom view of the device shown in FIG. 7A1. The skin electrodes (240) may be seen in relationship to the epilator blades (232) that rotate within drum (234).

FIG. 7B2 shows a bottom view of the device shown in FIG. 7A2. The skin electrodes (240) may be seen in relationship to the epilator blades (232) that rotate within drum (234). The electrodes (240) are wider in skin contact area than are those shown in FIGS. 7A1 and 7B2.

Our combination RF/mechanical epilator devices are of two generic circuit types: duo-pole devices and mono-pole devices. In the duo-pole devices, the RF energy is applied to the skin by a pair of electrodes adjacent the field of skin in which the hair strands are being mechanically pulled by the mechanical epilator component. In the mono-pole devices, the RF energy is applied to the skin through the epilator blades; the RF circuit is completed via a moving or stationery patch or electrode situated on the skin.

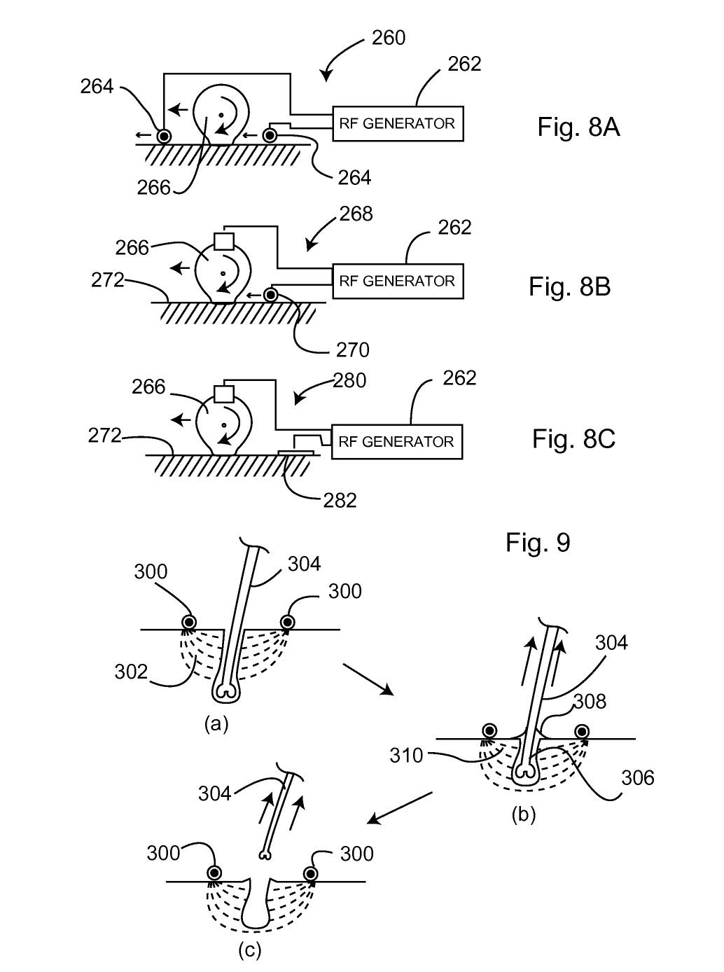

FIG. 8A shows a schematic representation of a dual-pole device, such as we describe with relation to certain of FIGS. 5A-6E₂ above. In this variation (260), RF generated by an RF source or generator (262) and applied to two electrodes (264) adjacent the epilation treatment area contacted by the mechanical epilation blades (266).

FIG. 8B shows a schematic depiction of a mono-pole device (268). In this variation, RF from the RF generator (262) is applied to the rotating epilator blades (266) and the circuit is completed by an electrode (270) situated against the skin (272). This variation may further have two variations. First, if the rotating epilator blades (266) are configured not to touch the skin during operation but, for instance, are spaced away from the skin and engage only the hair and the RF voltage is maintained at a non-arcing level, the effectiveness of the device is diminished since the hair shaft is a relatively poor conductor of RF current compared to the human skin. If the voltage to the blades is increased to a level allowing arcing to the skin, e.g., during the closest passage of the blades to the skin, that arcing occurs as the blades pinch the hair pulling adjacent skin upward, and the RF passes to that skin closely adjacent the hair. Such arcing delivers energy to the follicle tissue.

In another variation, the rotating epilator blades (266) are configured to touch the skin during the hair pinching portion of the blade rotation. The RF current is then applied directly to the skin very close to the site of the extended follicle and bulb. The rotating blades may be so-configured, e.g., by extending the diameter of the rotating blades (174) in the region of the blades that pinch the hair or by increasing the diameter of the blade region trailing that hair-pinch area. Obviously, lower RF voltage levels than those discussed above with relation to the “arcing” variation may be applied.

We have had good results with both of the variations shown in FIGS. 7A1, 7A2, 7B1, 7B2, 8A and 8B with continuously applied RF. The open area between the stationary electrodes may be, e.g., 2-6 cm². In this way and in most of the other variations described herein, the open area allows epilation or treatment of multiple hairs simultaneously.

FIG. 8C shows still another variation (280) of the monopole configuration. In this variation, the RF circuit is completed through the rotating epilator blades (266), through the skin (272) and is completed via a stationary patch (282) placed on the skin (272) desirably near the treatment area. In general, this latter configuration is least desirable due to the extended circuit length and increased power losses due to the length of that circuit.

FIG. 9 shows a schematic representation of our understanding of the effects of using our combination RF-mechanical epilator. Step (a) shows the application of RF energy between two electrodes (300). Also depicted is our understanding of the density of the energy flow (302) near to the surface of the skin. The energy concentration is generally considered to be more dense near the skin surface and then, more dense in the regions of the skin containing more ionic fluids, e.g., blood and sweat. The hair (304), in depicted step (a), has not yet been pulled towards the skin surface.

Step (b) in FIG. 9 shows the step of pulling the hair (304) and its attached follicle and bulb (306) up towards the skin surface and into the region (310) of higher RF energy density. This step may be carried out by a mechanical epilation device such as described above. This pulling step also creates a small hillock (308) at the skin surface. Application of an appropriate level of RF to the matrix (128 in FIG. 1) and other components of the follicle (collectively 114, 116 in FIG. 1) and hair bulb (102) heats those hair components, injures them, and is effective in causing a most-effective epilation effect.

Step (c) of FIG. 9 shows the complete removal of hair (304).

The RF may be applied to the skin in bursts coordinated with the active extension of individual hairs. Alternatively, RF energy may be continuously applied to the skin. RF energy may be applied to the skin using other timing sequences, for instance, the RF may be applied to the skin in the region of the hair to be extracted, at a time prior to that extraction to warm the area and to facilitate removal of the target hair.

RF operational parameters for our device generally fall within the values that follow. Of course, based upon the guidance provided herein, these parameters may be adjusted to achieve the results described herein. The RF carrier frequency may be 0.5 to 100.0 MHz., perhaps 1-10 MHz. Power levels may be up to about 30-35 Watts, although in most instances, a power level of 20 Watts is sufficient. The duty cycle may be between 5% and 100% (CW). The pulse length may be between about 1 msec and 1 second, typically 50-150 msec. The peak-to-peak voltage of the source may be between 80 and 1000 volts, perhaps between 150 and 600 volts, depending upon the load. For depilation, a voltage level of 200-100 volts, perhaps 300-400 volts (p-to-p), is a practical value. The RF pulse repetition rate may be between 0.5 and 200 Hz, e.g., about 50 and 150 Hz., typically at about 100 Hz.

Combination Light Source-Mechanical Epilator

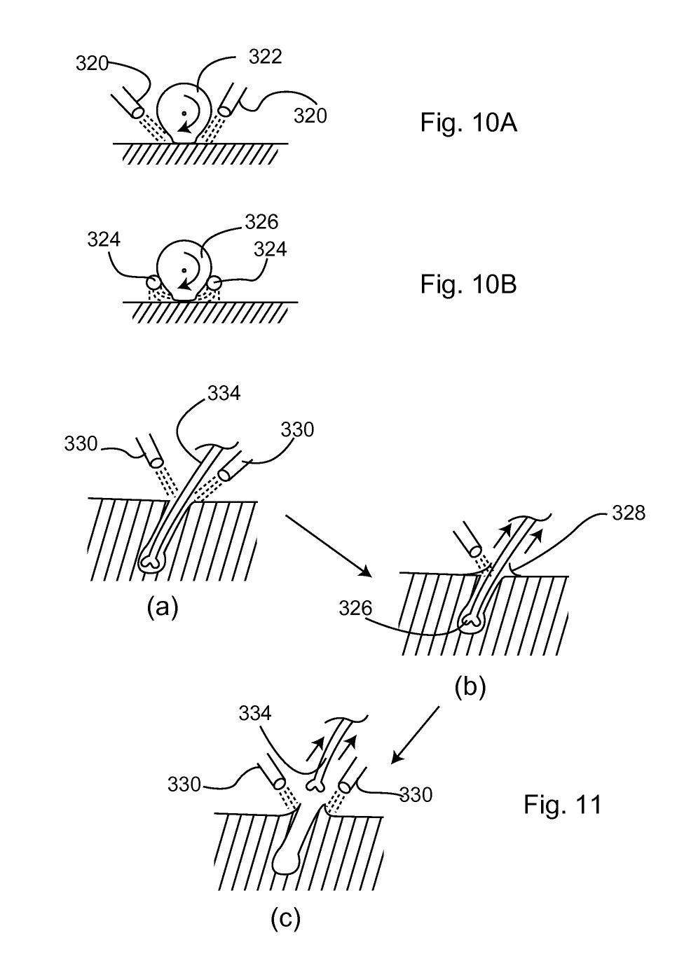

FIGS. 10A and 10B show two variations of our combination of a mechanical epilator component and, as its primary energy source, a light source component (the “epi-light”).

FIG. 10A shows our combination epilating device having a light source (e.g., laser or intensive pulsed light) (320) and an exempletive rotating epilator (322) serving as a mechanical epilator component. In the illustrated variation, the light sources (320) are spaced a short distance away from each location where a hair is to be extended from the skin by a mechanical epilator. One or more light sources (320) may be placed at other sites having such access to the skin surface.

The light sources (320) may, for instance, be lasers of sufficient intensity, perhaps with a lens or other optical device for focusing the emitted light energy or perhaps with a light transmission device (e.g., “light pipes,” prisms, mirrors (planar or focusing), etc.) allowing remote placement of the light sources (320). Other appropriately intense light sources, e.g., IPL, flash lamps, and the like, may also be used. In particular, light source (320) may comprise laser bars (including high power diode laser bars or HDB's) or laser stacks (such as are sold by OSRAM Opto Semiconductors GmbH and Jenoptik Aktiengesellschafft), or a series of individual laser diodes paralleling the axis of the rotating epilator (322).

The various light sources (320) may continuously illuminate or may intermittently illuminate the skin. One intermittent illumination variation may proceed with a timed or coordinated light pulse having a specific duration during which the hair is pulled upward towards the skin or may be pulsed at another timed interval. If the light source is pulsed at least to have a duration for the length of the skin extension, the hair bulb and lower part of the follicle will be in the “intense light field flux” area before light scatters in the skin. Typically, when the hair shaft is pulled upwardly is the lower part brought into this “light intense” area.

We believe the pulsed light sources should have a pulse length of between 5 and 300 msec, when coordinated with the rotational speed of the mechanical epilator. That is to say: if the mechanical epilator rotates at a rate of 1800 rpm, a pulse length of about 10 msec would be sufficient. The light energy fluence would be between about 5 and 80 Joule/cm². Typically between 15 and 35, perhaps between 5 and 20, Joule/cm² is adequate to heat the area but yet not burn the hair. These values will be adjusted depending upon the nature of the light source, e.g., its frequency, and skin tone. The listed values are suitable for an 800 nm pulsed diode laser. The spot size may be varied to cover the epilation region, e.g., about 1 cm. in diameter.

If the light sources continuously illuminate the skin, they may cause more pain, but have the advantage of pre-warming the upper part of the follicle prior to epilating it and thus causing the epilation itself to be more efficient and potentially less painful.

FIG. 10B shows a variation in which light sources (324) extend axially parallel to the axis of the rotating mechanical epilator (326).

FIG. 11 shows the procedure for using our combination light-mechanical epilation devices. This depicted process is similar to that shown in FIG. 9 with regard to the RF associated device. Step (a) shows the application of light energy from two light sources (300). Our understanding of the diffusion of the light energy density is that the density is greater near the surface of the skin, since light scatters beneath the skin surface. The hair (334), in this step (a), has not yet been pulled towards the skin surface.

Step (b) in FIG. 11 shows the step of pulling the hair (324) and its attached follicle and bulb (326) up towards the skin surface and into the region of higher light energy density. This step may be carried out by a mechanical epilation device such as described above. This pulling step also creates a small hillock (328) at the skin surface. This extension forming hillock (328) is brought into the region of high light flux. Application of an appropriate light level to the matrix (128 in FIG. 1) and other components of the follicle (collectively 114, 116 in FIG. 1) and bulb (102) heats those hair components, injures them, and is effective in causing a most-effective epilation effect. As noted above, the light source or sources (330) may remain illuminated or may be pulsed to, e.g., to the point of hair shaft (334) removal, as is shown in step (c).

Step (c) of FIG. 11 shows the complete removal of hair (334).

Combination HIFU-Mechanical Epilator

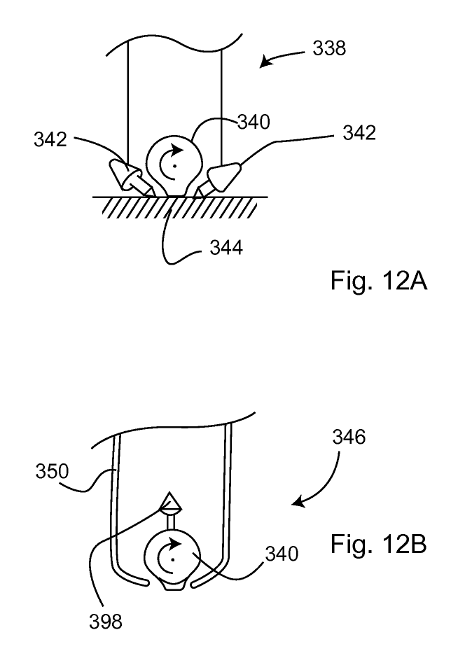

FIG. 12A provides a schematic side view of our combination ultrasound—mechanical epilator device, wherein the ultrasound source is the primary energy source.

By way of background, in the use of ultrasound in therapeutic applications, absorbed ultrasound energy changes the state of a target tissue area. In particular, ultrasound energy applied at high power densities can induce significant physiological effects on those tissues. These effects may result from either thermal or mechanical response of the tissue subjected to ultrasound energy. Thermal effects include hyperthermia and ablation of tissue. The absorption of ultrasound energy at the target area induces a sudden temperature rise, which causes coagulation or ablation of target area cells.

Generally, in therapeutic applications of ultrasound, it is important that the applied ultrasound energy causes the intended result solely at the target area without adversely affecting other tissue within the patient. A proper dose is delivered to the target area while the thermal and mechanical effects in intermediary and surrounding tissue are minimized. Proper focusing and control of High Intensity Focused Ultrasound (HIFU) is one of the primary criteria for successful therapeutic application of ultrasound.

U.S. Pat. No. 6,007,499, to Martin et al, and U.S. Pat. No. 6,042,556, to Beach et al, describe a focused ultrasonic transducer used for HIFU hyperthermia treatments. The intensity of ultrasonic waves generated by the focused transducer increases from the source to the region of focus, at which a very high temperature may be achieved. The absorption of the ultrasonic energy at the focal region induces a sudden temperature rise of affected tissue and causes an irreversible ablation of the target volume of cells.

U.S. Pat. No. 5,092,336, to Fink, describes a device for localization and focusing of acoustic waves in tissues. The procedure is known as time-reversed acoustics, and is also described in an article by Fink, entitled, “Time-reversed acoustics,” Scientific American, November 1999, pp. 91-97. In this procedure, a target is enclosed by an array of transducers that delivers an unfocused acoustic beam on a reflective target in a medium, for example, a site in organic tissue. Reflected signals from the target detected by ultrasound transducers in a regular array outside the patient are stored, the distribution in time and the shapes of the echo signals are time-reversed, and the reversed signals are applied to the respective transducers of the array. In most cases, the target constitutes a secondary source, which reflects or scatters a wave beam applied to it.

U.S. Pat. No. 6,161,434 to Fink et al., describes methods to use time-reversed acoustics to search for a faint sound source. U.S. Pat. No. 5,428,999 to Fink, describes methods for detecting and locating reflecting targets, ultrasound echographic imaging, and concentrating acoustic energy on a target.

PCT Patent Publication WO 97/29699 to Ben-Haim, entitled, “Intrabody energy focusing,” describes methods for optimizing irradiation of a target area of the body by using a radiation-sensing probe inserted into the body. U.S. Pat. No. 5,590,657 to Cain et al., describes a HIFU system including a phased array of ultrasound transducers located outside the patient. Methods for refocusing the beam are described. U.S. Pat. No. 6,128,958 to Cain, describes an architecture for driving an ultrasound phased array.

Returning to FIG. 12A, our combination ultrasound source-mechanical depilator device (338) comprises an exempletive rotary epilating component (340) serving as a mechanical epilator component and one or more focused ultrasonic sources (342). The ultrasonic sources (342) may be of the designs discussed just above. The ultrasonic sources (342) are aimed towards the root (344) of the hair so to allow the ultrasonic energy from the multiple sources to merge at the hair root and to cause harm to that hair structure.

As is the case with the other primary energy sources, the ultrasound energy may be applied to the skin in bursts that may be coordinated with the active extension of individual hairs by the mechanical epilator. Alternatively, ultrasound energy may be continuously applied to the skin. Ultrasound energy may be applied to the skin using other timing sequences. For instance, the ultrasound may be applied to the skin in the region of the hair to be extracted, at a time prior to that extraction to warm the area and to facilitate removal of the target hair.

FIG. 12B shows another variation of our combination ultrasound source-mechanical depilator device (346) comprises a rotary epilating component (340) serving as a mechanical epilator component and one or more ultrasonic sources (348) coupled to that rotary epilating component (340) and, in turn, the hair to be extracted. The ultrasonic sources (348) may be of the designs discussed just above or that shown in Published U.S. Patent Application No. 2007/0173746. The ultrasonic source or sources (348) are indirectly coupled to the hair to allow the ultrasonic energy from the source to cause harm to that hair structure. Alternatively, the transducer may be placed in the rotating epilating component (340) and allowed to rotate with the epilating component (340) and coordinated to emit ultrasonic waves as that component (340) grasps the hair shaft.

Combination RF-Light-Mechanical Epilator

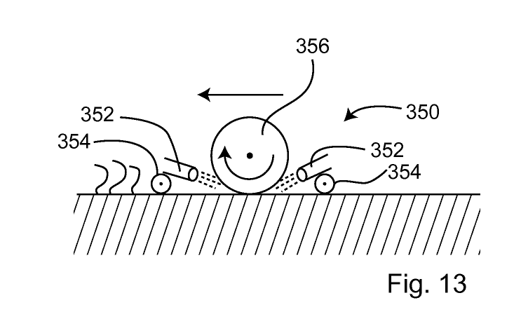

FIG. 13 schematically depicts a variation of our device, in particular, the variation comprises a mechanical epilator in combination with a primary energy source, an RF source, and an adjunct energy source, a light source. In particular, FIG. 12 shows a schematic view of our combination epilating device (350) having one or more light sources (e.g., laser or intensive pulsed light) (352), an RF source (via electrodes 354), and an exempletive rotary epilating component (356) serving as a mechanical epilator component. In the illustrated variation, the light sources (352) are associated with electrodes (354). One or more light sources (352) may be placed at other sites having such access to the skin surface. One or more light sources (352) may be added to each of the RF electrodes (352).

Another variation comprises a mechanical epilator in combination with a primary energy source, an RF source, and an adjunct energy source, an ultrasound source. The adjunct ultrasound source or sources may be situated with respect to the rotary epilating component as are the adjunct light sources shown in FIG. 13. The ultrasound source or sources may be associated with the RF electrodes or placed at other sites having such access to the skin surface.

In general, our device may comprise a mechanical epilator, a primary energy source selected from the group consisting of at least one RF, light, and ultrasound sources, and an optional adjunct energy source selected from the group consisting of at least one RF, light, and ultrasound sources.

Hair Region Treatment

Our devices have a further variation in which the hair shaft is not necessarily pulled from the skin but, instead, one or more of the hair shaft, the components of the hair and surrounding skin are treated or affected by the primary energy sources of our device. These variations of our device comprise an epilator component that pulls the hair towards the surface of the skin and pull the attached skin components upward towards the device forming the hillocks mentioned elsewhere, but the epilator component is configured to release the hair shaft before its physical removal. In this variation, our combination device may injure the follicular components and cause the hair later to fall out and to inhibit or slow further hair growth. The function of releasing the hair before extraction is the major change from our other hair extraction variations discussed here.

Other Combinations

Although we have explained the mechanical components for lifting the hair shaft and follicle using an epilator based on a rotary mechanical tweezer, our device may comprise other mechanical hair removal devices, a primary energy source selected from the group consisting of RF, light, and ultrasound sources, and an optional adjunct energy source selected from the group consisting of RF, light, and ultrasound sources. Other such mechanical structures include:

So-called electric shavers (e.g., such as the Braun “lift and cut” mechanism)

So-called ultrasound electric shavers (e.g., as marketed by Braun). The ultrasound application is said to lift the follicle and hair shaft during operation.

Manual shavers (e.g., Mach3 Fusion razors). These manual razors having multiple blade construction are said to lift the hair shaft during use thus resulting in a smoother shave.

In each instance, the mechanical hair removing structures may be combined with the primary energy emitting components as described above emitting pulsed or continuous energy and secondary components, as desired.

A variation of our device comprises our primary energy-emitting components, specifically our light source in isolation, our RF source in isolation, our ultrasound source in isolation (with or without the optional adjunct components) in a configuration suitable for skin treatment subsequent, e.g., as much as 5-40 minutes later, to independent mechanical epilation procedures. Ephemeral blood remaining in the follicle opening is a site for absorbing light, RF, or ultrasound energy. Such treatment will also inhibit later hair growth or hair growth rate.

A cooling or chilling component, e.g., such as found in the Philips Satinelle Ice Premium, may be included in our combinations to provide a trailing cooling function and to alleviate initial pain from the mechanical epilation step. Vibrator components may also be used as adjuncts to our combination devices to assist in epilation.

### EXAMPLES

### Example 1

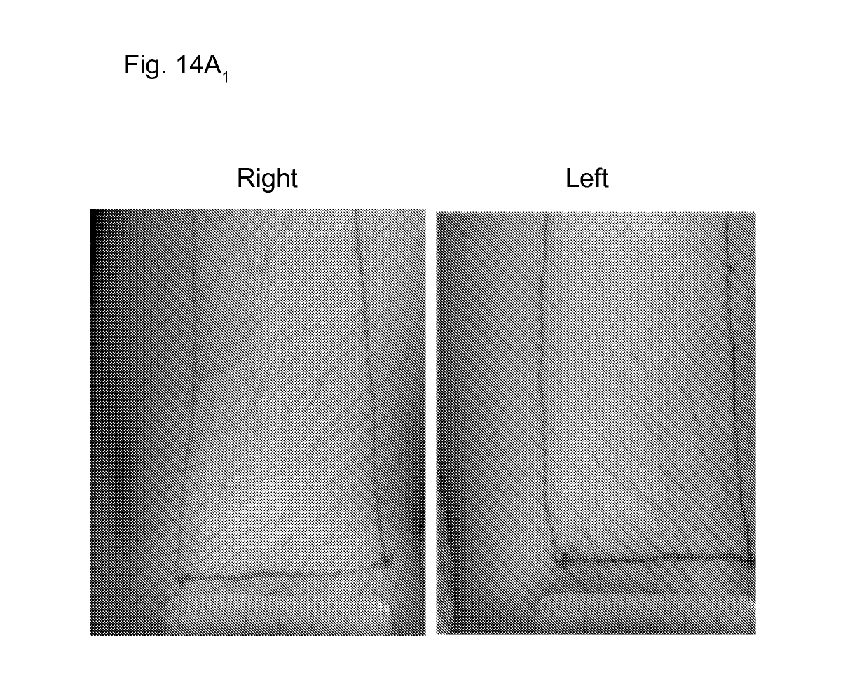

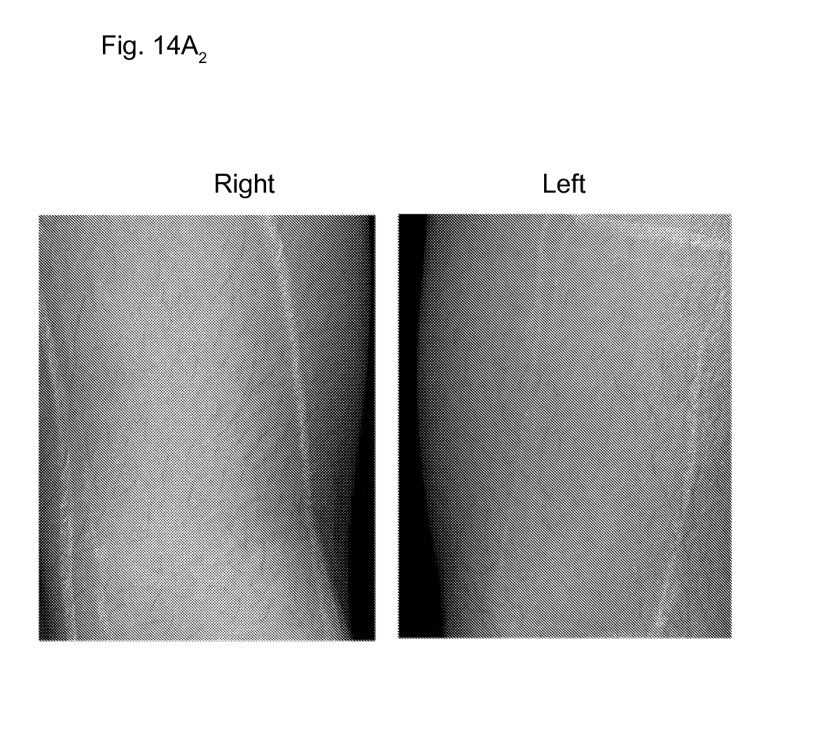

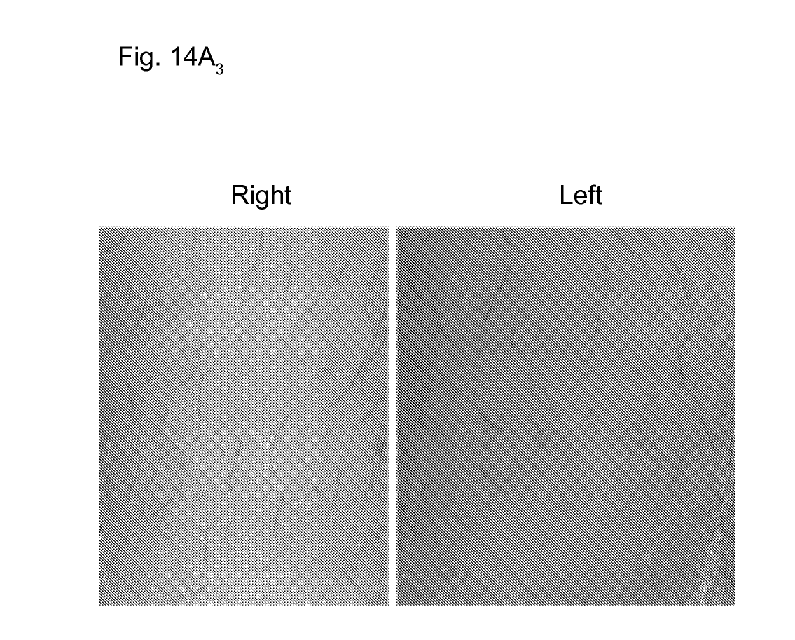

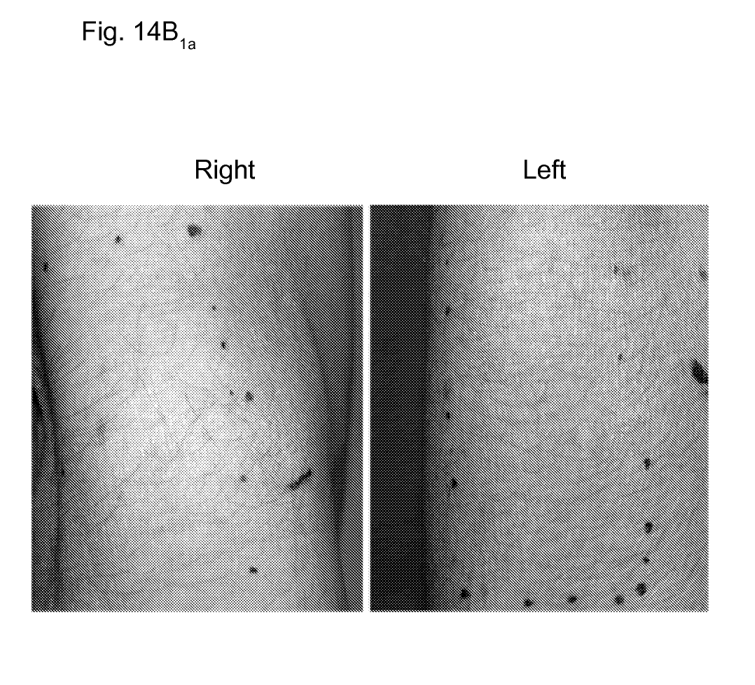

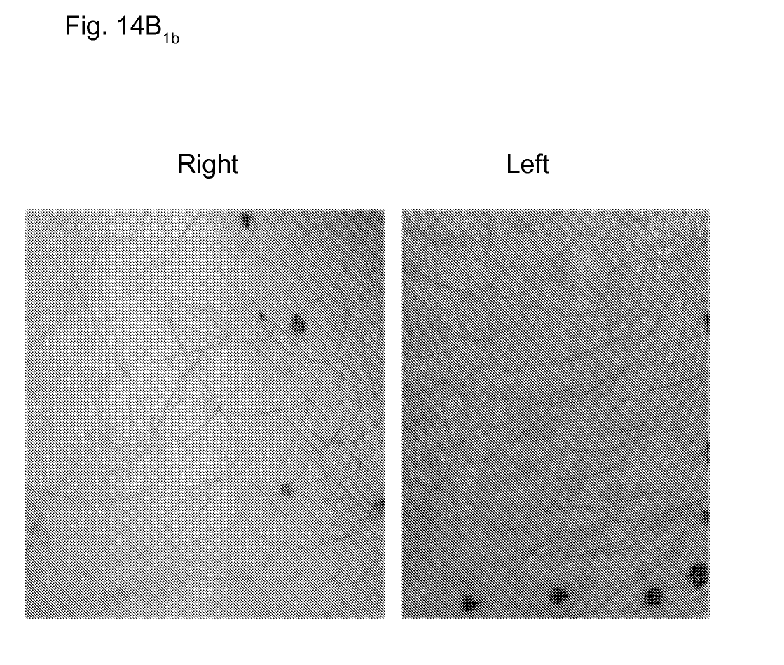

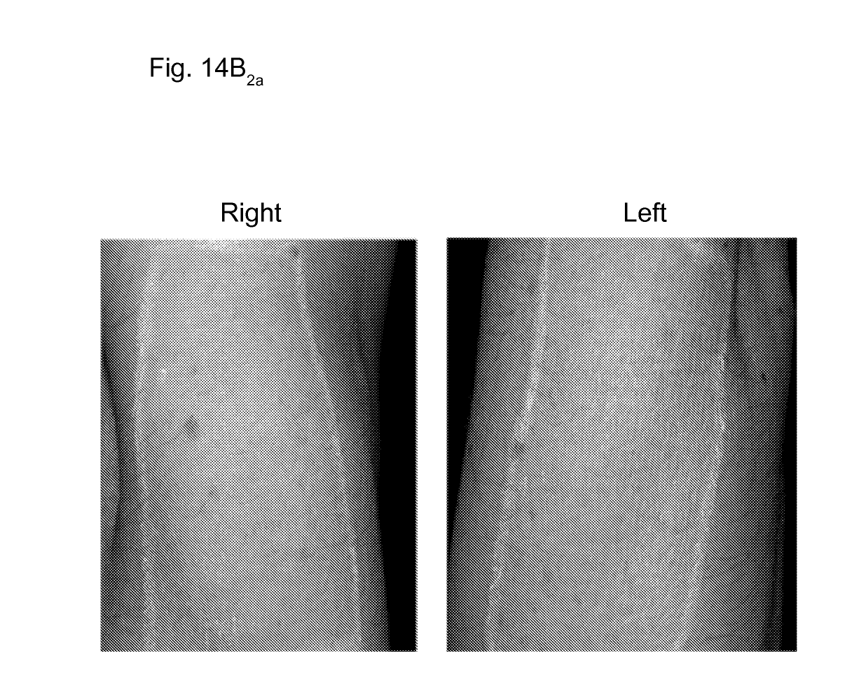

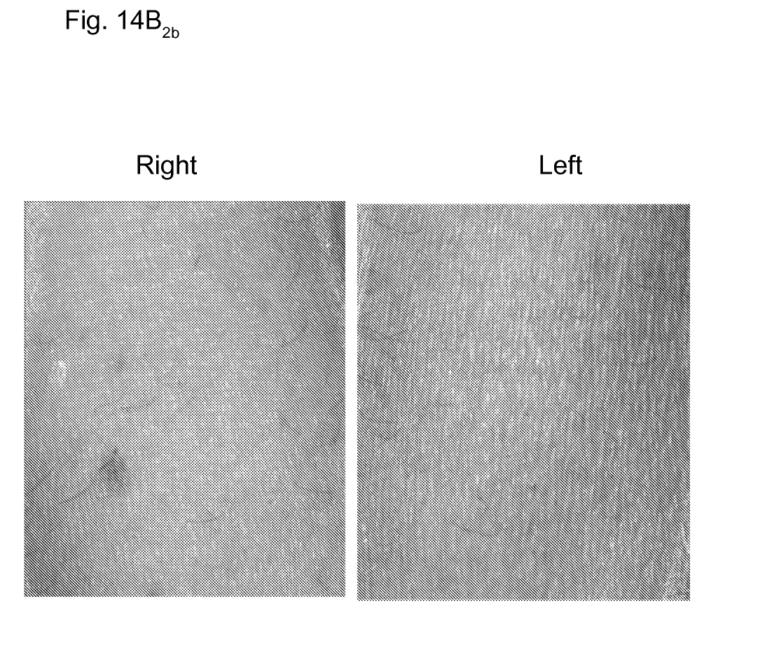

The arms of two female subject individuals (mother and daughter) were treated with one variation of our RF-energy emitting (RF-epi) device to qualitatively check the effectiveness of our combination epi-RF device. The two subjects were also treated with a mechanical epilator not having an RF emitter, as a comparison. The right arm of each subject was treated with the conventional epilator; the left arm of each individual was treated with our RF-epi device. Photographs of each of the treated areas of the arms are shown in FIG. 14. The arm areas are shown before treatment of any kind and three weeks after the treatments. The RF parameters were—a pulse repetition rate of 100 Hz., 60% duty cycle, a carrier frequency of 1 MHz., peak-to-peak voltage was about 400 volts, the power supply was rated at 20 Watts, and the treatment was for 60 seconds.

| FIG. 13 | | |
|---|---|---|
| Photo number | content of photo | time of photo |
| A1 | daughter - right | before treatment |
| | and left arm | |
| A2 | daughter - right | three weeks |
| | and left arm | after treatment |
| A3 | daughter - right | three weeks |
| | and left arm | after treatment |
| | close-up | |
| B1a | mother - right | before treatment |
| | and left arm | |
| B1b | mother - right | before treatment |
| | and left arm | |
| | close-up | |
| B2a | mother - right | three weeks |
| | and left arm | after treatment |
| B2b | mother - right | three weeks |
| | and left arm | after treatment |
| | close-up | |

In each of photos A2, A3, B2a, and B2b, the left arms treated with our RF-epi device had less hair than did the right arms treated with a conventional epilator.

### Example 2

A male subject individual was also treated with a conventional epilator, our epi-RF device upon moistened skin, and our epi-RF device with dry skin. For esthetic and comparative observation, the test individual also shaved an area, but did not use any epilator there.

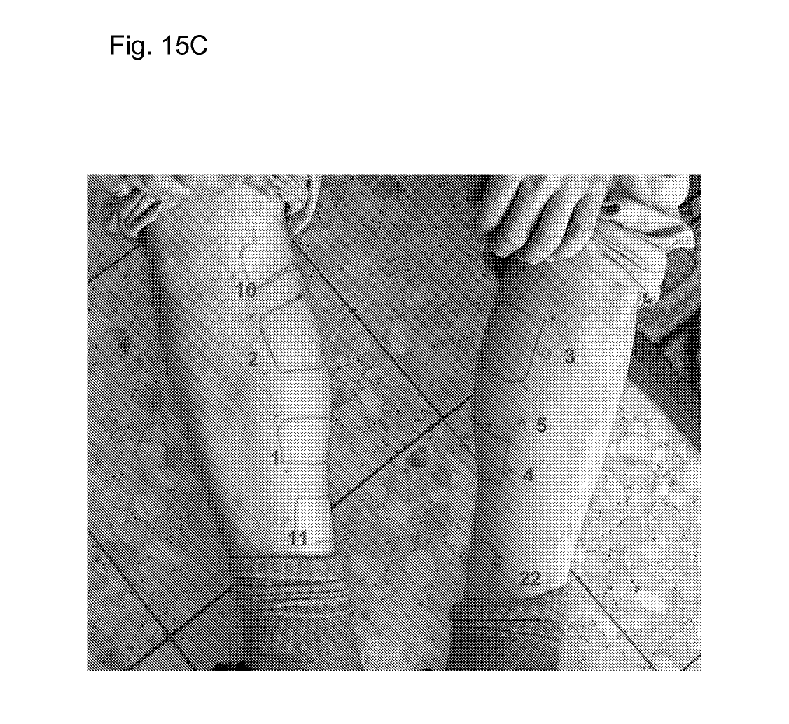

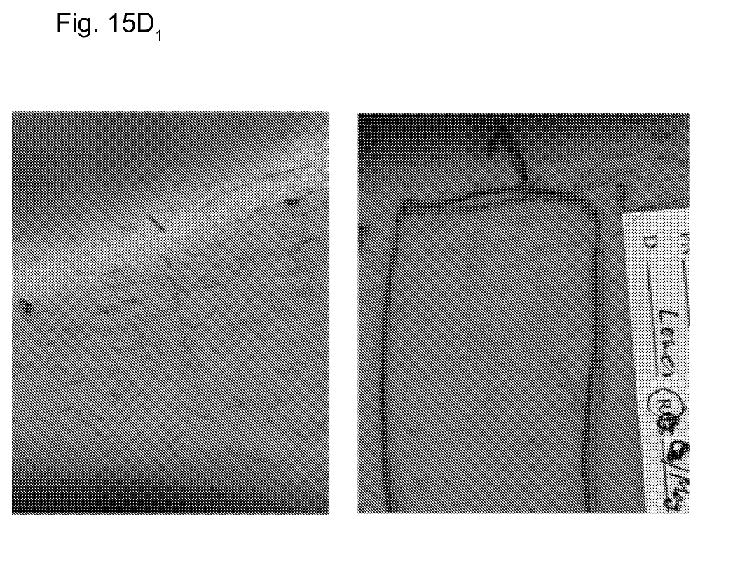

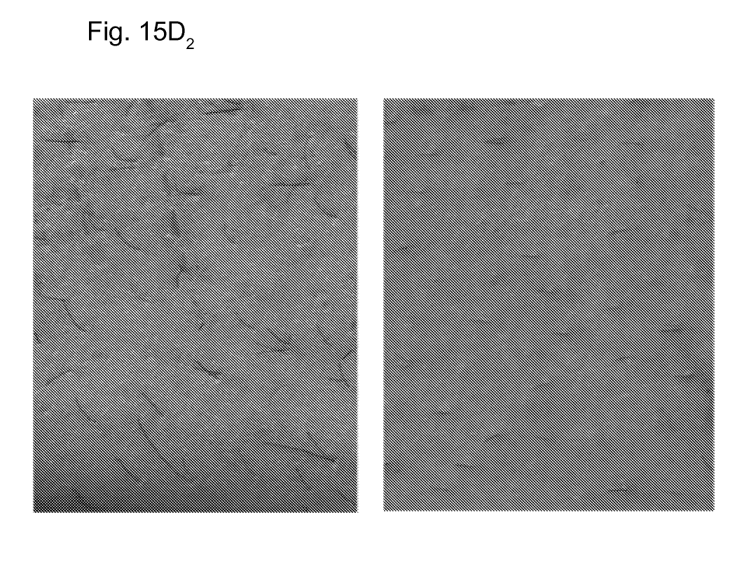

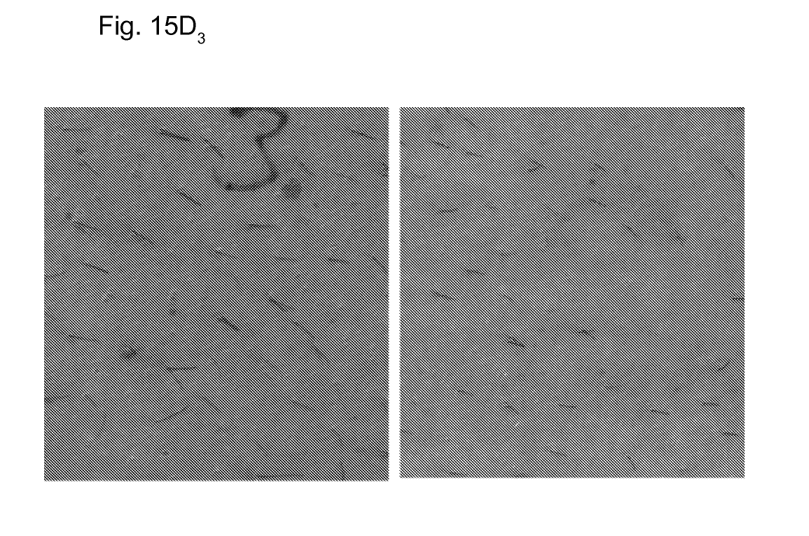

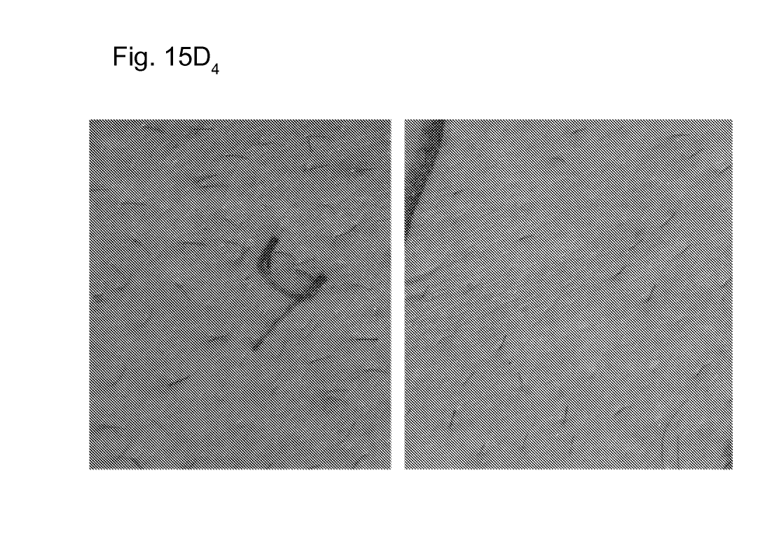

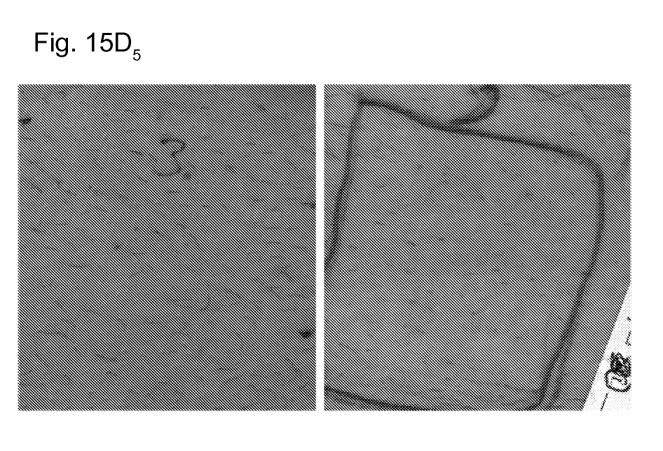

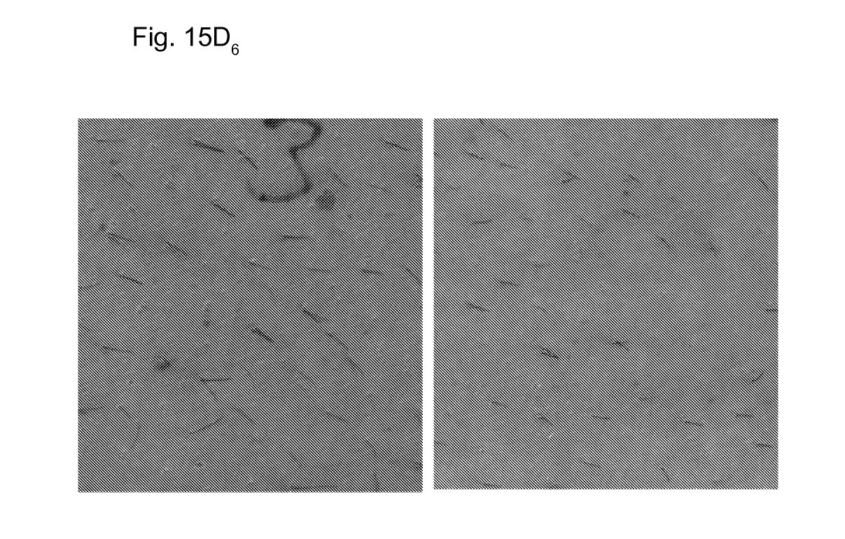

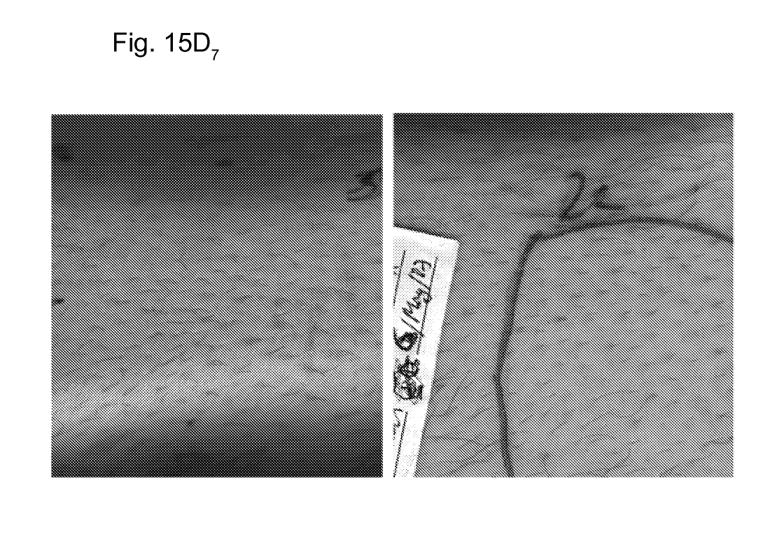

Each area was treated twice, an initial treatment and a second treatment about four weeks later. The photos in FIG. 15 show the subject's skin before any treatment and after nine weeks. The RF parameters: 20 watts for the wet skin on the first treatment, 0-20 watts for the first treatment on dry skin. The second treatment utilized 2 watts for each type of treatment. The electrodes were 4 cm. apart.

Photo C shows the numbered areas on the individual corresponding to the photos.

| FIG. | | | |
|---|---|---|---|
| 15 | | | |
| Photo | Skin | | photo |
| number | Area | device | type |
| D1 | 1 | conventional | normal |
| | | epilator | |
| D2 | 1 | conventional | close-up |
| | | epilator | |
| D3 | 2 | epi-RF (w/ | normal |
| | | dry skin) | |
| D4 | 4 | epi-RF (w/ | close-up |
| | | dry skin) | |
| D5 | 3 | epi-RF (w/ | normal |
| | | wet skin) | |
| D6 | 3 | epi-RF (w/ | close-up |
| | | wet skin) | |
| D7 | 22 | shaver | normal |

In the photos taken nine weeks after initial treatment, those skin patches treated with our epi-RF device and seen in each of photos D3, D4, D5, and D6 (D5 and D6 being of the same area of skin) showed significantly less hair than the skin area treated with the conventional epilator. The shaved skin patch (photo D7) appeared substantially unchanged during the test.

We have provided what we believe to be the most reasonable explanation of the various physical phenomenon we have observed, however we do not wish to be bound to those theories in the claims expressed below, unless we specifically refer to those theories.

We claim as our invention:

1. A device for removing hair from a body comprising in combination:
a mechanical epilator component configured to grasp hair shafts and pull attached hair follicles and hair roots towards the skin surface and to pull the hair shafts out of the skin, and
at least one primary energy source configured to apply energy to the follicular region of such hair shafts as they are pulled, before they are pulled, or after they are pulled by the mechanical epilator component.

2. The device of claim 1 wherein the at least one primary energy source is configured to apply energy to the skin.

3. The device of claim 1 wherein the at least one primary energy source is configured to apply energy to the hair shafts.

4. The device of claim 1 wherein the at least one primary energy source comprises a continuous or pulsed light source.

5. The device of claim 1 wherein the at least one primary energy source comprises one or more continuous or pulsed laser bars, high power diode laser bars, laser stacks, or series of individual laser diodes.

6. The device of claim 1 wherein the at least one primary energy source comprises a continuous or pulsed RF source.

7. The device of claim 6 wherein the at least one primary energy source comprises a pulsed RF source where the RF pulse duration is between 1 msec and 1 sec, the carrier frequency is between 0.5 MHz and 40 MHz and the duty cycle is between 1 and 99%.

8. The device of claim 1 wherein the at least one primary energy source comprises a continuous or pulsed ultrasound source.

9. The device of claim 8 wherein the at least one primary energy source comprises a pulsed ultrasound source having a pulse duration between 10 msec and 2 seconds.

10. The device of claim 1 further comprising an adjunct energy source selected from an RF source, a light source, an ultrasound source, and an heat source.

11. The device of claim 1 further comprising a cooling component configured to cool the skin surface in the region of the hair shafts before or after they are pulled out by the mechanical epilator component.

12. The device of claim 1 wherein the at least one primary energy source is configured to apply energy to the follicular region of such hair shafts as they are pulled by the mechanical epilator component.

13. A device for removing hair from a body comprising in combination:
a mechanical rotary epilator component configured to grasp hair shafts, to pull attached hair follicles and hair roots towards the skin surface, and to pull the hair shafts out of the skin during rotation, and
at least one primary energy source configured to apply energy to the follicular region of such hair shafts after they are pulled out by the mechanical rotary epilator component.

14. The device of claim 13 wherein the at least one primary energy source is configured to apply energy to the skin.

15. The device of claim 13 wherein the at least one primary energy source is configured to apply energy to the hair shafts.

16. The device of claim 13 wherein the at least one primary energy source comprises a continuous or pulsed light source.

17. The device of claim 13 wherein the at least one primary energy source comprises one or more continuous or pulsed laser bars, high power diode laser bars, laser stacks, or series of individual laser diodes.

18. The device of claim 13 wherein the at least one primary energy source comprises a continuous or pulsed RF source.

19. The device of claim 18 wherein the at least one primary energy source comprises a pulsed RF source where the RF pulse duration is between 1 msec and 1 sec, the carrier frequency is between 0.5 MHz and 40 MHz and the duty cycle is between 1 and 99%.

20. The device of claim 13 wherein the at least one primary energy source comprises a continuous or pulsed ultrasound source.

21. The device of claim 20 wherein the at least one primary energy source comprises a pulsed ultrasound source, having a pulse duration between 10 msec and 2 seconds.

22. The device of claim 13 further comprising an adjunct energy source selected from an RF source, a light source, an ultrasound source, and an heat source.

23. The device of claim 13 further comprising a cooling component configured to cool the skin surface in the region of the hair shafts before or after they are pulled out by the mechanical epilator component.

24. The device of claim 13 wherein the at least one primary energy source is configured to apply energy to the follicular region of such hair shafts as they are pulled by the mechanical epilator component.

25. A device for removing hair from a body comprising in combination:
a mechanical rotary epilator component configured to grasp hair shafts and pull attached hair follicles and hair roots towards the skin surface and to pull the hair shafts out of the skin during rotation, and
at least one primary continuous or pulsed RF energy source configured to apply energy to the follicular region of such hair shafts as they are pulled by the mechanical rotary epilator component.

26. The device of claim 25 wherein the at least one primary energy RF source comprises a continuous source.

27. The device of claim 25 wherein the at least one primary energy RF source comprises a pulsed source.

28. The device of claim 25 further comprising an adjunct energy source selected from an RF source, a light source, an ultrasound source, and an heat source.

29. The device of claim 25 wherein the at least one primary energy RF source comprises electrodes located adjacent the mechanical rotary epilator component.

30. The device of claim 29 wherein the electrodes located adjacent the mechanical rotary epilator component are rollers.

31. The device of claim 29 wherein the electrodes located adjacent the mechanical rotary epilator component are stationary.

32. The device of claim 25 further comprising a cooling component configured to cool the skin surface in the region of the hair shafts after they are pulled out by the mechanical rotary epilator component.

33. A device for removing hair from a body comprising in combination:
a mechanical rotary epilator component configured to grasp hair shafts and pull attached hair follicles and hair roots towards the skin surface and to pull the hair shafts out of the skin during rotation, and
at least one primary continuous or pulsed light energy source configured to apply energy to the follicular region of such hair shafts as they are pulled by the mechanical rotary epilator component.

34. The device of claim 33 wherein the at least one primary energy light source comprises a continuous source.

35. The device of claim 33 wherein the at least one primary energy light source comprises a pulsed source.

36. The device of claim 33 further comprising an adjunct energy source selected from an RF source, a light source, an ultrasound source, and an heat source.

37. The device of claim 33 wherein the at least one primary energy light source comprises one or more continuous or pulsed laser bars, high power diode laser bars, laser stacks, or series of individual laser diodes located adjacent the mechanical rotary epilator component.

38. The device of claim 33 further comprising a cooling component configured to cool the skin surface in the region of the hair shafts after they are pulled out by the mechanical rotary epilator component.

39. A device for affecting hair in a body comprising in combination:
a mechanical epilator component configured to grasp hair shafts and pull attached hair follicles and hair roots towards the skin surface, and
at least one primary energy source configured to apply energy to the follicular region of such hair shafts as they are pulled by the mechanical epilator component.

40. The device of claim 39 wherein the at least one primary energy source is configured to apply energy to the skin.

41. The device of claim 39 wherein the at least one primary energy source is configured to apply energy to the hair shafts.

42. The device of claim 39 wherein the at least one primary energy source comprises a continuous or pulsed light source.

43. The device of claim 39 wherein the at least one primary energy source comprises one or more continuous or pulsed laser bars, high power diode laser bars, laser stacks, or series of individual laser diodes.

44. The device of claim 39 wherein the at least one primary energy source comprises a continuous or pulsed RF source.

45. The device of claim 39 wherein the at least one primary energy source comprises a continuous or pulsed ultrasound source.

46. A method for removing hair from skin having hair, comprising the step of:
contacting the device of claim 1 to the skin, and
moving the device across the skin,
thereby removing hair from the skin.

47. A method for removing hair from skin having hair, comprising the step of:
contacting the device of claim 13 to the skin, and
moving the device across the skin,
thereby removing hair from the skin.

48. A method for removing hair from skin having hair, comprising the step of:
contacting the device of claim 25 to the skin, and
moving the device across the skin,
thereby removing hair from the skin.

49. A method for removing hair from skin having hair, comprising the step of
contacting the device of claim 33 to the skin, and
moving the device across the skin,
thereby removing hair from the skin.

50. A method for affecting hair in skin having hair, comprising the step of:
contacting the device of claim 39 to the skin, and
moving the device across the skin,
thereby affecting hair in the skin.
